(19) United States
(12) Patent Application Publication
(10) Pub. No.: US 2022/0171919 A1
(43) Pub. Date: Jun. 2, 2022

(54) METHODS AND SYSTEM FOR RECORDING USER INPUTS WHEN INTERACTING WITH A USER INTERFACE

(71) Applicant: CERNER INNOVATION, INC., KANSAS CITY, KS (US)

(72) Inventors: Pravat Santra, Bangalore (IN); Suhas B N, Bangalore (IN); Siddharth P Shanbhogue, Bangalore (IN); Sunil Bansal, Bangalore (IN)

(21) Appl. No.: 17/108,093

(22) Filed: Dec. 1, 2020

(51) Int. Cl. G06F 40/174; G06F 9/455; G06F 16/958; G16H 10/60; G06F 21/62; G06F 9/451

(52) U.S. Cl. CPC G06F 40/174; G06F 9/45529; G06F 9/451; G16H 10/60; G06F 21/6218; G06F 16/986

(57) ABSTRACT

Methods and systems are provided for collecting and recording any inputs that a user provides to an application or website while the user is interacting with the user interface. The collected information is recorded and stored either in the user's browser cache or sent back to the application backend. The storage location is determined by the application developer or website developer. The recorded data may be automatically restored when the user accesses the location where the user inputs were collected from. The user's data is stored only in the user cache or the application backend such that the data provided by the user is safe and secure.

### BACKGROUND

When interacting with websites and web-based applications, it is common for a user to provide information in the form of inputs into fillable fields within the application or website. Many web-based applications and websites require users to fill in multiple forms or multiple fillable fields within the application or website. On occasion, websites and applications require a large list of input requirements such as personal information and medical histories. These inputs by the user may be required in order to proceed within the application or website. When working with large forms a large amount of user entries, users may be prone to face interruptions, disconnections, or errors during the process of providing the required inputs. For instance, when working on pages with multiple input fields, users can potentially experience issues that require them to refill the pages all over again. This may occur due to user error (accidentally clicking the back button or submitting the form too soon), network issues, or issues in the page validations. Another possible scenario is when a user does not have all the information required to submit the form and wants to come back to the form to fill it in at a later time. Leaving the form page within the application or website would cause the user to lose all information already filled. Additionally, some websites or applications may require a user to input the same information each time the application or website is used. Thus the user would need to re-input the same information each time the page or form is used. In such cases as described above, the users replicate their inputs multiple times and spend considerable time and effort in doing so. An effective solution has not been proposed or implemented, as set forth hereinafter.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims.

In brief and at a high level, this disclosure describes, among other things, methods, systems, and computer-readable media for collecting and recording any inputs that a user provides to an application or website while the user is interacting with the user interface. The collected information is recorded and stored either in the user's browser cache or sent back to the application backend. The storage location is determined by the application developer or website developer. The recorded data may be automatically restored when the user accesses the location from which the user inputs were collected. The user's data is stored only in the user cache or the application backend such that the data provided by the user is safe and secure. In some embodiments, the system and methods may automatically detect the presence of a form within an application and detect user input within fillable fields present in the form. Once user input is detected, the system and methods may automatically record the user input and transmit it back to the application backend or in a local browser cache.

### BRIEF DESCRIPTION OF THE DRAWINGS

Embodiments are described in detail below with reference to the attached drawing figures, wherein:

FIG. 1 is a block diagram of an exemplary computing system suitable to implement embodiments of the present invention;

FIG. 2 is a diagram of an exemplary architecture for a method and system useful for storing user input information from a live web-based form automatically in accordance with an embodiment of the present invention;

FIG. 3 is a flow diagram showing a method for storing user input information; and

FIG. 4 is a diagram showing a schematic of database storage.

### DETAILED DESCRIPTION

The subject matter of the present invention is described with specificity herein to meet statutory requirements. However, the Description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described. In the same vein, the claimed subject matter might also be embodied in other ways, to include different components or combinations of components, similar to those described in this document, in conjunction with other present or future technologies.

When interacting with websites and web-based applications, it is common for a user to provide information in the form of inputs to the application. Many of these websites or applications require users to provide input into multiple forms to proceed within the website or application. Some of these forms are large with many required inputs. These forms are, at times, lengthy and difficult to complete without error or interruption. Users of these forms may experience some degree of interruption, disconnection, or error. In one example, while working on pages with multiple input fields, users can experience issues that require them to refill the pages over again. This may occur due to user error such as pressing the back button prematurely or pre-mature submission of the form. Additionally, network issues or page validation issues may present difficulties in completion of a form in a single sitting. In some situations, it is not possible to complete a form as the user may not possess all of the required information and may need to revisit the site or application to finish the form at a later time. In all of these scenarios the user would lose all of the information input into the forms thus requiring the user to re-input the required information into the form fields. Other applications or websites may have users that re-use a page or form multiple times and require re-input of identical information into the form fields. This replication of inputs costs the user considerable time and effort. Additionally, the re-input of identical information presents the possibility of introducing error into the inputs. This time and effort cost in addition to the potential errors poses a significant problem when forms and pages require large amounts of input.

The Live web-based form concept, as described by the present invention, collects and records any and all inputs that a user provides within a website page or a web-based application. This collection and recordation of inputs occurs when the user is interacting with the user interface of an application such that the user inputs information into form fields within the application. The collected information is then stored in either the user's browser cache or sent to the application backend to be stored on a server. The recorded data can then be automatically restored when the user accesses the page again. Upon revisiting the page or form, the user's previous entries are restored to the location of the prior user input. Since the data collected is only stored on the user's local cache or the application backend server, the data is safe and secure from potential corruption.

In a first embodiment, a system useful for storing user input information from a live web-based form automatically is provided. The system comprises computer readable media having executable instructions stored thereon which may be executed by a processor. The instructions cause the processor to identify a form within an application or website, the form being a user modifiable form having form fields where the user may input information. The system may then identify the form fields and detect user input into the application in one of the form fields that are detected. The user's input data is then transmitted to either a user's local cache or a server to be stored. Once the data is stored on either the cache or the server, the data is automatically restored to the form fields when the user access the form again.

In another embodiment, one or more computer storage media having computer-usable instructions are provided. When the computer-usable instructions of the computer storage media are used by one or more computing devices, the one or more computing devices perform a method for storing user input information from a live web-based form automatically. The method causes the processor to identify a form within an application or web-site, the form being a user modifiable form having form fields where the user may input information. The system may then identify the form fields and detect user input into the application in one of the form fields that are detected. The user's input data is then transmitted to either a user's local cache or a server to be stored. Once the data is stored on either the cache or the server, the data is automatically restored to the form fields when the user access the form again.

In yet another embodiment, a computerized method for storing user input information from a live web-based form automatically is provided. The method is performed by an application server. The method causes the processor to identify a form within an application or website, the form being a user modifiable form having form fields where the user may input information. The system may then identify the form fields and detect user input into the application in one of the form fields that are detected. The user's input data is then transmitted to either a user's local cache or a server to be stored. Once the data is stored on either the cache or the server, the data is automatically restored to the form fields when the user access the form again.

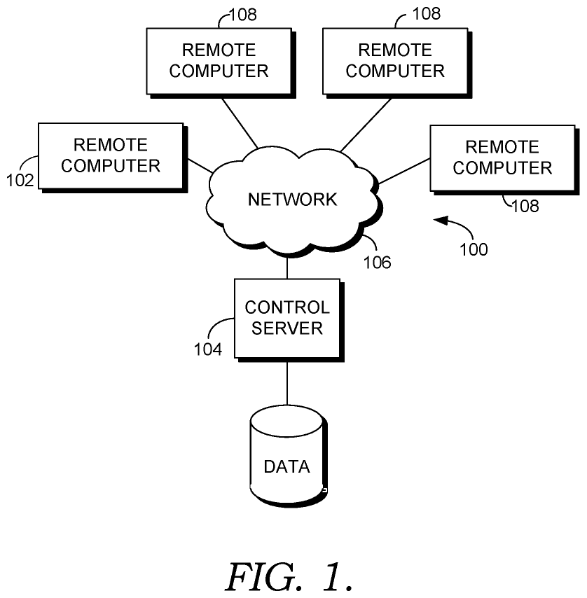

Referring now to the drawings in general, and initially to FIG. 1 in particular, an exemplary computing system environment, for instance, a healthcare information and management system, in which embodiments of the present invention may be implemented is illustrated and designated generally as reference numeral 100. It will be understood and appreciated by those of ordinary skill in the art that the illustrated medical information computing system environment 100 is merely an example of one suitable computing environment and is not intended to suggest any limitation as to the scope of use or functionality of the invention. Neither should the healthcare information and management system be interpreted as having any dependency or requirement relating to any single component or combination of components illustrated therein.

The present invention may be operational with numerous other general-purpose or special-purpose computing system environments or configurations. Examples of well-known computing systems, environments, and/or configurations that may be suitable for use with the present invention include, by way of example only, personal computers, server computers, handheld or laptop devices, multiprocessor systems, microprocessor-based systems, set top boxes, programmable consumer electronics, network PCs, minicomputers, mainframe computers, distributed computing environments that include any of the above-mentioned systems or devices, and the like. In embodiments, the present invention may be implemented in computing system environments employed within healthcare facilities, such as a distributed network that communicatively couples multiple, affiliated hospitals and/or related outpatient clinics. For example, computing systems employed for healthcare facility implementation may include, in addition to those examples of well-known computing systems, patient monitoring devices, scanning devices, infusion pumps, ventilators, and the like.

The present invention may be described in the general context of computer-executable instructions, such as program modules, being executed by a computing device. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in local and/or remote computer storage media including, by way of example only, memory storage devices.

With continued reference to FIG. 1, the exemplary healthcare information and management system includes a general-purpose computing device in the form of a computer server, illustrated as server 102. The server 102 may be employed within the healthcare information and management system. Components of the server 102 may include, without limitation, a processing unit, internal system memory, and a suitable system bus for coupling various system components, including a database or database cluster. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA,®) local bus, and Peripheral Component Interconnect (PCI) bus, also known as Mezzanine bus.

The server 102 typically includes, or has access to, a variety of computer-readable media, for instance, a computer store 104. Computer-readable media can be any available media that may be accessed by server 102, and includes volatile and nonvolatile media, as well as removable and non-removable media. By way of example, and not limitation, computer-readable media may include computer storage media and communication media. Computer storage media may include, without limitation, volatile and nonvolatile media, as well as removable and non-removable media, implemented in any method or technology for storage of information, such as computer-readable instructions, data structures, program modules, or other data. In this regard, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server 102. Computer storage media does not comprise signals per se. Communication media typically embodies computer-readable instructions, data structures, program modules, or other data in a modulated data signal, such as a carrier wave or other transport mechanism, and may include any information delivery media. As used herein, the term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, RF, infrared, and other wireless media. Combinations of any of the above also may be included within the scope of computer-readable media.

The computer storage media discussed above and illustrated in FIG. 1, including a computer store 104, provides storage of computer-readable instructions, data structures, program modules, and other data for the server 102. As such, the server 102 may be programmed to perform various tasks using computer-readable instructions, including embodiments of methods described hereinafter. For example, the server 102 may be programmed with one or more modules that facilitate management of specific tasks in a workflow of a healthcare information and management system. Exemplary modules might include a task generation module configured to populate a workflow with one or more tasks in response to receipt of a medical order, and a form assignment module configured to assign an initial form to a therapeutic agent associated with or specified in a medical order. Further exemplary modules might include a form compatibility module configured to identify an initial form assigned to a medical order, determine whether the current form of a therapeutic agent is compatible with an initial form, and when it is determined that the current form of the therapeutic agent is not compatible with the initial form, issue an error. As used herein, “compatible” refers to interchangeability of one form for another, such that patient care outcome is not negatively affected, or such that any difference in patient care outcome due to the difference of two forms is clinically negligible. In further terms, compatible forms may be considered interchangeable and/or acceptable substitutions for one another by clinicians or other users in a clinical setting.

In embodiments, the server 102 is coupled to a computer store 104 such that the server 102 may access, communicate with, and otherwise retrieve information stored by the computer store 104. In embodiments, the computer store 104 is a database configured to store information encoded as data. In some embodiments, the computer store 104 is configured to permanently store data such as electronic medical records for a plurality of patients. As such, the computer store 104 includes memory. In another embodiment, the computer store 104 is configured to temporarily store data, such that the computer store 104 may act, at least partially, as a cache for faster data access and retrieval by the server 102. Additionally or alternatively, the computer store 104 includes long-term permanent data storage for storing one or more electronic medical records (EMR) of patients associated with a medical entity (e.g., hospital, group of hospitals, physicians group, an outpatient clinic). In embodiments, the computer store 104 includes computer-readable media, as previously described hereinabove. In further embodiments, the computer store 104 may comprise a form compatibility database that is configured to store a compatibility index of one or more forms for each of a plurality of therapeutic agents, for example.

The server 102 may operate in a distributed network environment 106 of the healthcare information and management system 100. The server 102 and the distributed network environment 106 use logical connections to communicate with one or more remote computers 108. Remote computers 108 may be located at a variety of locations in a medical or research environment, for example, but not limited to, clinical laboratories, hospitals and other inpatient settings, veterinary environments, ambulatory settings, medical billing and financial offices, hospital administration settings, home healthcare environments, and clinicians' offices. Clinicians may include, but are not limited to, a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; genetic counselors; researchers; veterinarians; students; and the like. The remote computers 108 may also be physically located in non-traditional healthcare environments so that the entire healthcare community may be capable of integration with the distributed network environment 106.

The remote computers 108 may include a handheld device or mobile device, in some embodiments. The remote computers 108 may include, incorporate, and/or be coupled to a scanning device, such as barcode scanners, radio frequency identification (RFID) reading devices, or real-time locating system (RTLS) devices, for example. Scanning devices may be handheld devices, in some embodiments. As such, remote computer 108 may be a scanning device configured to read machine-readable identifiers that encode information which is used to specifically and/or uniquely identify a healthcare worker, a patient, a medical device, and a therapeutic agent associated with a medical order. The remote computer 108 including a scanning device may be used to electronically document the completion of workflow tasks within a clinical setting when said scanning device is used to identify and track healthcare workers, patients, medical devices, and therapeutic agents within the clinical setting, as associated with workflow tasks. Exemplary remote computer 108 may include personal computers, servers, routers, network PCs, peer devices, other common network nodes, or the like, and may include some or all of the components described above in relation to the server 102. The devices may be personal digital assistants or other like devices, in some embodiments.

Continuing, exemplary distributed network environment 106 may include, without limitation, local area networks (LANs) and/or wide area networks (WANs). Such networking environments are commonplace in offices, enterprise-wide computer networks, intranets, and the Internet. When utilized in a WAN networking environment, the server 102 may include a modem or other means for establishing communications over the WAN, such as the Internet. In a networked environment, program modules or portions thereof may be stored in the server 102, in the computer store 104, or on any of the remote computers 108. For example, and not by way of limitation, various application programs may reside on the memory associated with any one or more of the remote computers 108. It will be appreciated by those of ordinary skill in the art that the network connections shown are exemplary and other means of establishing a communications link between the computers (e.g., server 102 and remote computers 108) may be utilized.

Although many other internal components of the server 102 and the remote computers 108 are not shown, those of ordinary skill in the art will appreciate that such components and their interconnection are well known. Accordingly, additional details concerning the internal construction of the server 102 and the remote computers 108 are not further disclosed herein.

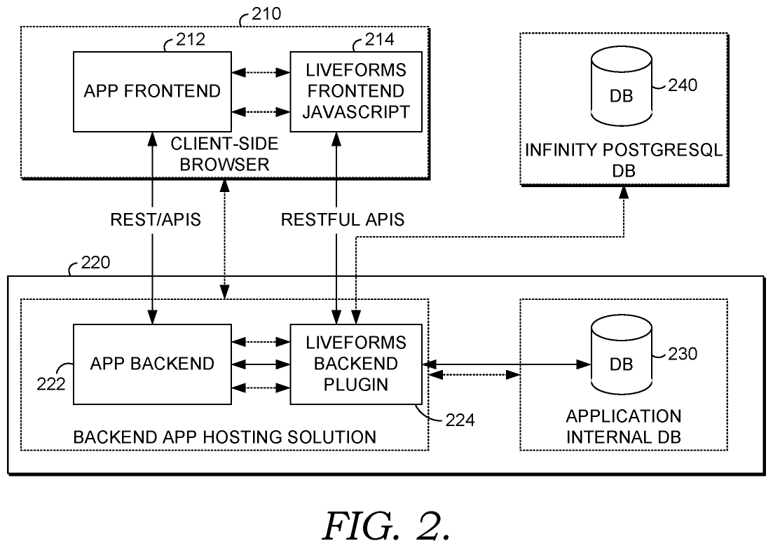

Turning now to FIG. 2, a diagram is provided illustrating a sample architecture for an application which may store user input data automatically in either a user's local cache or an application's internal data base. The location of the data store can be configured by application developer teams such that the data storage location can be configured in the host application's configuration. It should be understood that this and other arrangements described herein are set forth only as examples. Other arrangements and elements (e.g., machines, interfaces, functions, orders, groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, various functions may be carried out by a processor executing instructions stored in memory.

The sample architecture environment of FIG. 2 includes a client-side browser 210 that provides a presentation layer for formatting and delivery of information to the application backend 222 or application layer for further processing or display. The client-side browser 210 receives and processes all the requests (sometimes in the form of event messages) to execute user code on the virtual computer system. In one embodiment, the client-side browser 210 serves as a front door to all the other services provided by a virtual computer system. The client-side browser 210 processes the requests and makes sure that the requests are properly authorized. For example, the client-side browser 210 may determine whether the user associated with the request is authorized to access the user code specified in the request. The client-side browser 210 may receive a request to execute such user codes in response to JavaScript requests from a user. Also, any information (e.g., headers and parameters) included in the JavaScript request may also be processed and utilized when executing the user code. Any other protocols, including, for example, HTTP, MQTT, and CoAP, may be used to transfer the message containing the code execution request to the client-side browser 210.

The user code as used herein may refer to any program code (e.g., a program, routine, subroutine, thread, etc.) written in a specific program language. In the present disclosure, the terms “code,” “user code,” and “program code,” may be used interchangeably. Such user code may be executed to achieve a specific task, for example, in connection with a particular web application or mobile application developed by the user. For example, the codes may be written in JavaScript (node.js), Java, Python, and/or Ruby. The request may include the code (or the location thereof) and one or more arguments to be used for executing the user code. For example, the user may provide the user code along with the request to execute the user code. In another example, the request may identify a previously uploaded program code (e.g., using the API for uploading the code) by its name or its unique ID.

The code for the application frontend 212 serves as the code associated with the client-side browser 210 portion of an application. Code for the frontend 212 converts data provided into a graphical interface. For example the code for the frontend 212 can be programmatically configured to facilitate displaying a graphical user interface for an application or website. Such a graphical user interface (GUI) may contain fillable forms such that a user may input information into the form. These fillable forms may contain fillable fields within the form such that the user may input information into the fillable fields. The GUI may also display a web page of a website such that the web page contains fillable fields within the web page or form within the web page. These fillable forms caused to be displayed by way of the GUI may contain multiple fillable fields.

As an example fillable form, a GUI may display a fillable form which form may contain multiple fillable fields within the form. Each of the fillable fields may receive user input to provide information within the field. These fields may be provided for the user to provide information. The fillable fields within the form may provide a space for a user to input information such as user information, patient information, patient vitals, feedback, or any other information. Some example forms may require some of the fields within the form to be filled out in order to proceed within the application or the website. Some of the forms may not be required to proceed within the website. For example, a user may be required to input user identification credentials in order to proceed to the next page within the application but may be provide a fillable field to provide optional feedback.

The sample architecture environment of FIG. 2 includes an application backend 220 that provides a data access layer for simplified access to data stored in persistent storage of some kind. The backend 220 sorts and arranges data and ensures that everything on the server side of an application or website is functioning correctly. The code associated with the application backend 222 serves as the code associated with the backend 210 portion of an application. The code for the application backend 222 may utilize python, PHP, Ruby, Perl, Node.js, or compiled languages like C #, of backend code 222 to communicate with the backend 220 portion of the application. For example the backend code 222 can be programmatically configured to facilitate storing data on a server, creating libraries, and working with system components.

Live web-based forms frontend code 214 serves as instructions to implement the frontend portion of the present invention. A file containing the Live web-based forms frontend code 214 is injected into the frontend code of the application by the developer. The Live web-based forms frontend code 214 may be in the form of a JavaScript which when injected into the code for the frontend 212 may cause client-side browser 210 to perform the instructions contained within Live web-based forms frontend code 214. The Live web-based forms frontend code 214 may also be in the form of HTTP, MQTT, and CoAP requests. The Live web-based forms frontend code 214 may contain instructions executable which when executed by the client-side browser 210 hook into the document object module of the client-side browser 210. Once the Live web-based forms frontend code 214 hooks into the document object module, the client-side browser 210 may then make application program interface (API) calls to the backend to perform its functionalities. The API calls may conform to representational state transfer (REST) set of constraints. Such API calls or RESTful APIs allow the frontend or backend to access and manipulate textual representations of the application or website displayed on the GUI. For example, the RESTful APIs allow the client-side browser 210 to identify data and send such data to the backend 220 for storage in a server. The functionalities of the Live web-based forms frontend code 214 includes identification of a user of the application, data collection from the user's input into the fillable forms within the GUI, and data storage. The Live web-based forms frontend code 214 may determine identification of the user of the application. The user identification may also be determined by the identification mechanism employed by the application.

Live web-based forms backend plugin 224 serves as instructions to implement the frontend portion of the present invention. Live web-based forms backend plugin 224 is added in the development system of the application within the backend 220. Live web-based forms backend plugin 224 is added as an additional package and allows the developer of the application or website to gain access to a set of APIs that are exposed by the package for handling data. The live web-based forms backend plugin 224 may be in the form of a Python script which when imported into the application backend code 222 may cause backend 220 to perform the instructions contained within backend plugin 224. The Live web-based forms backend plugin 224 may also be in the form of PHP, Ruby, Perl, Node.js, or compiled languages like C #, of Java code scripts. The Live web-based forms backend plugin 224 may contain executable instructions which when executed by the backend 220 allows for the management and handling of data collected by the client-side browser 210 and Live web-based forms frontend code 214. The Live web-based forms backend plugin 224 may be hosted on separate endpoints or may use the existing application itself.

The Live web-based forms backend plugin 224 is designed to facilitate the processing and storage of the user input from the fillable form fields. Such storage may be accomplished in an internal data store 230, external datastore 240, or user cache. The developer of the application may cause the Live web-based forms backend plugin 224 to store the received data from the fillable forms into one of the many storage options. For example, the Live web-based forms backend plugin 224 may be configured to receive data from the client-side browser 210. Once the data is received from the client-side browser 210 the backend may be configured to store the data from the fillable forms into an internal database. Such a database may be on an application server such that the application developers maintain direct control of the database. In an additional embodiment, the backend 220 may be configured to store the data from the fillable forms into an infinity database such as an Infinity PostgreSQL database.

In an additional embodiment, the client-side browser 210 is configured to store the received data from the fillable forms into a local cache on a user's browser or local server. For example, a user may input information in the fillable forms displayed on the GUI. Once the information is input into the fillable forms or the fillable fields within an application or website, the data is received by the client-side browser 210. The received data is then stored on a local cache on the user's local browser or server. Once the user closes the application, the fillable forms or fillable fields within the application is cleared. As the user re-visits the application or website by opening or starting it, the data stored in the local cache for each fillable field is restored to its respective fillable field.

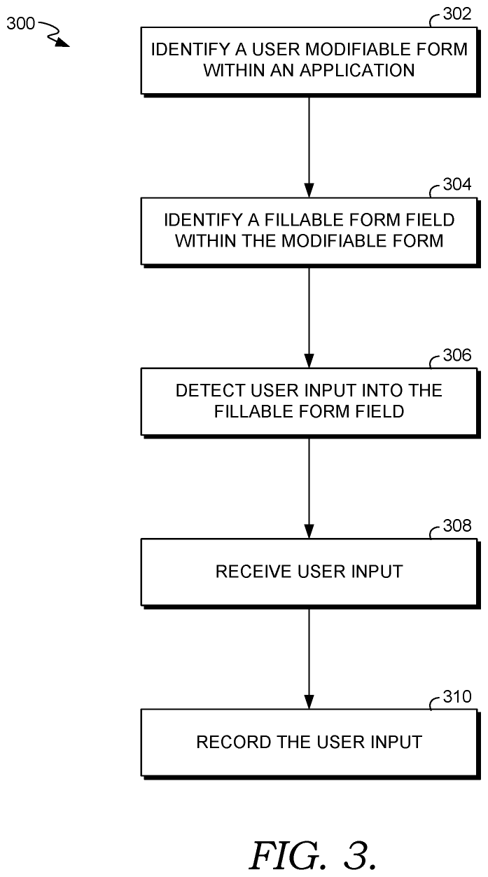

Turning to FIG. 3, a flow diagram is provided illustrating a method 300 for storing user input information from a live web-based form automatically is provided, in accordance with an embodiment of the present invention. In some embodiments, the method 300 may be performed using one or more computer storage media, such as previously described. For example, computer storage media may have computer-usable instructions that, when used by one or more computing devices, cause said computing devices to perform the method 300.

At block 302 the method is used to identify a user modifiable form within an application or website. A user modifiable form may comprise a form or page that comprises at least a section where a user may input information. Such an input may be manually input or input using a text to speech system. Once the modifiable form is identified within the application page or webpage, block 304 identifies a fillable field within the modifiable form. The fillable field may be a space where the user may click or select to designate and input user information into the field. For example, upon opening the application, the system determines that there exists a modifiable form comprising multiple fields that a user may input information. The user may input information into any of the fields that may be modified. The fields may exist as a way to gather information in response to a question or a desire to know information about the user such as personal information or demographic information. These fields may be required to be filled in order to proceed within the application.

At block 306, the method detects user input into the user modifiable fields. The user input may be a user typing to input information or may be a user selection from a drop-down menu in response to a question. Additionally, user input may be a verbal response recorded via a microphone in connection with the user device. Other types of user input may be used in connection with detecting user input into the fillable form field. At block 308, the user input may be received from the user device to the application hosting server. Once received, block 310 records the user input into a location for future use. The user input may be recorded in a user's local cache, an application server, or a cloud-based location such as an infinity database. Any other location that may be suitable but not discussed herein may be used to store the user input information.

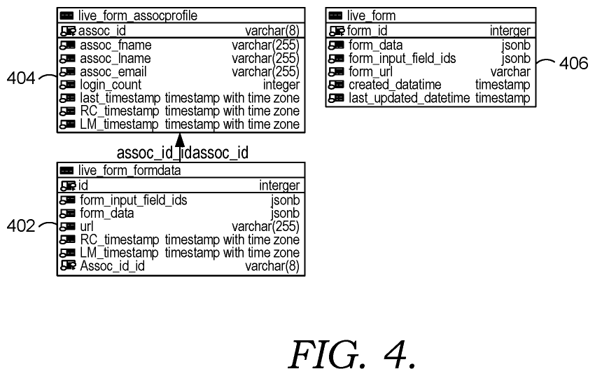

Turning now to FIG. 4, an exemplary schematic for usage within the Live web-based form system architecture. FIG. 4 shows an example of some information and the way that information may be stored for later use. The information saved may be in a form that contains a time stamp. Such a time stamp may include a date and a time that the user input the information. The time stamp may include the time zone of the location of the user. The user location information may also be input included along with the user information.

Continuing with FIG. 4, to access the form a user may be required to identify themselves such that the application may store the input information associated with the user. A user may input user identification information into the application form. The application may also use the identification information employed by the host application. The user identification may be stored with the user input information. User identification information may include but is not limited to first name, last name, username, password, codename, permission identification, or any other identifying information that may be required by the form or application.

As an example use of the live web-based form system, a user opens a form to input information, the form being integrated into an application or a webpage. The user, as an example, may be a healthcare worker, a clinician, or any other user of the application or webpage. Once the form is opened, the application may identify the user and store that information in a table. Some forms within a healthcare system may require a healthcare worker or clinician with appropriate credentials to complete and sign the form before it is released into an electronic medical record (EMR). The Live web-based form system may identify the user as a healthcare worker with appropriate credentials. This identification may be stored in a table such as table 402. Table 402 is managed by the backend plugin 224 and the information stored on table 402 is dependent on the application. Table 402 may store user identification information such as user ID, form data, URL of the webpage, timestamp, associate ID, credentials, authorization, and other identifying information.

Once the user is identified, additional background information of the user may be stored in table 404. Such information stored in table 404 may be user ID, user full name, user first name, user last name, user email, login count, last timestamp, current timestamp, and other background information. Upon inputting information into form fields within the form, such information may be stored in table 406. Live web-based form table 406 may store detailed information about the input information. Information stored in table 406 may be form ID, form data, form input field, form URL, created datetime, last updated datetime timestamp, or other information about the form and form fields. Information about the user and form inputs is stored in tables 402, 404, and 406 while the user inputs information. This allows the user or healthcare worker to leave a workstation with the form not being complete and unsigned. The healthcare worker can then return and finish filling out the form without losing already input information. Once the form is completed it may be signed by the healthcare worker with appropriate credentials and released into an EMR.

Many variations can be made to the illustrated embodiment of the present invention without departing from the scope of the present invention. Such modifications are within the scope of the present invention. Other modifications would be readily apparent to one of ordinary skill in the art, but would not depart from the scope of the present invention.

From the foregoing it will be seen that this invention is one well adapted to attain all ends and objects hereinabove set forth together with the other advantages which are obvious and which are inherent to the structure. It will be understood that certain features and subcombinations are of utility and may be employed without reference to other features and subcombinations. This is contemplated by and is within the scope of the invention.

Since many possible embodiments may be made of the invention without departing from the scope thereof, it is to be understood that all matter herein set forth or shown in the accompanying drawings is to be interpreted as illustrative of applications of the principles of this invention, and not in a limiting sense.

1. A system for storing user input information from a live web-based form automatically, the system comprising:
a processor; and
computer readable media having computer-executable instructions stored thereon which, when executed by the processor, cause the processor to:
identify a user modifiable form within an application, the user modifiable form having one or more fillable form field candidates;
identify the one or more fillable form fields candidates within the user modifiable form;
receive identification credentials for a user;
based on the identification credentials, authorize the user to input information into the user modifiable form;
detect user input in at least one of the one or more fillable form field candidates within the user modifiable form;
receive, from the at least one of the one or more fillable form field candidates, the user input;
automatically record the user input for the at least one of the one or more fillable form field candidates;
transmit and store the recorded user input; and
following the processor transmission of the recorded user input to a local cache and the fillable form being closed, automatically restore the recorded user input from the local cache into the fillable form field candidates in the user modifiable form upon reopening of the user modifiable form.

2. The system of claim 1, wherein the user input is further stored in a server.

3. The system of claim 1, wherein the user input is stored in a local cache.

4. The system of claim 1, wherein the user input is further stored in a cloud-based database.

5. The system of claim 4, wherein the cloud-based database is an infinity database.

6. The system of claim 1, wherein a system code is injected into a frontend code of the application.

7. The system of claim 6, wherein the system code is a javascript file which hooks into a document object management system of the user device.

8. The system of claim 1, wherein the computer readable media is added as an additional package import into an application backend system.

9. The system of claim 8, wherein the additional package import is added as a Python file.

10. One or more computer storage media having computer-usable instructions that, when used by one or more computing devices, cause the one or more computing devices to perform a method for storing user input information from a live web-based form automatically, the method comprising:
identifying a user modifiable form within an application, the user modifiable form having one or more fillable form field candidates;
identifying the one or more fillable form field candidates within the user modifiable form;
receiving identification credentials for a user;
based on the identification credentials, authorizing the user to input information into the user modifiable form;
detecting user input in at least one of the one or more fillable form field candidates within the user modifiable form;
receiving, from the at least one of the one or more fillable form field candidates, the user input;
automatically recording the user input for the at least one of the one or more fillable form field candidates and the identification credentials;
transmitting and storing the automatically recorded user input; and
following the processor transmission of the recorded user input to a local cache and the fillable form being closed, automatically restoring the recorded user input from the local cache into the at least one of the one or more fillable form field candidates in the user modifiable form upon reopening of the user modifiable form.

11. The media of claim 10, wherein the user input is stored in a server operated by a system administrator.

12. The media of claim 10, wherein the user is identified as a first user with one or more credentials to access the form.

13. The media of claim 12, wherein the user is a healthcare worker and the form is accessed within a healthcare system.

14. The media of claim 13, wherein the form is released into an electronic medical record upon completion of all required form fields.

15. The media of claim 10, wherein a system code is injected into a frontend code of the application.

16. The media of claim 15, wherein the system code is a JavaScript file which hooks into a document object management system of the user device.

17. A computerized method for storing user input information from a live web-based form automatically is provided, in accordance with an embodiment of the present invention, the method comprising:
identifying a user modifiable form within an application, the user modifiable form having one or more fillable form field candidates;
identifying the one or more fillable form field candidates within the user modifiable form;
receiving identification credentials for a user;
based on the identification credentials, authorizing the user to input information into the user modifiable form;
detecting user input in at least one of the one or more fillable form field candidates within the user modifiable form;
receiving, from the at least one of the one or more fillable form field candidates, the user input;
automatically recording the user input for the at least one of the one or more fillable form field candidates and the identification credentials;
transmitting and store the automatically recorded user; and
following the processor transmission of the recorded user input to a local cache and the fillable form being closed, automatically restoring the recorded user input from the local cache into the at least one of the one or more fillable form field candidates in the user modifiable form upon reopening of the user modifiable form.

18. The method of claim 17, wherein the user input is stored in a server.

19. The method of claim 17, wherein the user input is stored in a local cache.

20. The method of claim 17, wherein the user input is stored in a cloud based database.
